(19) United States
(12) Patent Application Publication
(10) Pub. No.: US 2020/0009085 A1
(43) Pub. Date: Jan. 9, 2020

(54) Methods For Treating Ovarian Cancer

(71) Applicant: RADIUS HEALTH, INC., Waltham, MA (US)

(72) Inventor: Gary Hattersley, Stow, MA (US)

(21) Appl. No.: 16/336,358

(22) PCT Filed: Sep. 27, 2017
(86) PCT No.: PCT/US2017/053834

§ 371 Date: Mar. 25, 2019

Related U.S. Application Data
(60) Provisional application No. 62/400,495, filed on Sep. 27, 2016.

(51) Int. Cl. A61K 31/138; A61K 9/00; A61K 31/337; A61K 31/519; A61K 31/506; A61P 35/00

(52) U.S. Cl. CPC A61K 31/138; A61K 9/0053; A61P 35/00; A61K 31/519; A61K 31/506; A61K 31/337

(57) ABSTRACT

Methods of treating ovarian cancer are disclosed.

### PRIORITY CLAIM

This application claims the benefit of U.S. Provisional Application No. 62/400,495, filed Sep. 27, 2016, which is incorporated herein by reference in its entirety.

### BACKGROUND

Ovarian tumors are known to often express estrogen receptor (ER) including ERα. ERs can be activated by estrogen and translocate into the nucleus to bind to DNA, thereby regulating the activity of various genes. See, e.g., Marino et al., “Estrogen Signaling Multiple Pathways to Impact Gene Transcription,” Curr. Genomics 7(8): 497-508 (2006); and Heldring et al., “Estrogen Receptors: How Do They Signal and What Are Their Targets,” Physiol. Rev. 87(3): 905-931 (2007). Ovarian cancers may be of different types including epithelial, stromal and sarcomas. Epithelial ovarian cancers are typically divided into serous, endometrioid, mucinous, clear cell and undifferentiated. Ovarian cancers may also be primary peritoneal, stromal and sarcomas. Regardless of origin or type, ovarian cancer is a serious and often fatal affliction with a great recognized need for new and effective treatment modalities.

### BRIEF SUMMARY OF THE INVENTION

Certain embodiments of the methods disclosed herein relate to treating ovarian cancer and/or tumor in a subject comprising administering to the subject a therapeutically effective amount of RAD1901.

Certain embodiments of the methods disclosed herein relate to treating ovarian cancer and/or tumor in a subject comprising administering to the subject a therapeutically effective amount of RAD1901 of RAD1901 and a therapeutically effective amount of one or more second therapeutic agents.

In certain embodiments of the methods disclosed herein, the ovarian cancer and/or tumor treated expresses ERα.

### . BRIEF DESCRIPTION OF DRAWINGS AND TABLES

FIG. 1: Tumor growth inhibition and regression effects of RAD1901 (p.o. daily at 30 mg/kg or 60 mg/kg) to PDX models (mice with established ovarian patient derived xenograft) tumors).

FIG. 2: Individual tumor growth inhibition and regression effect at day 32 in PDX models treated with RAD1901 (p.o., daily at 30 mg/kg or 60 mg/kg).

### DETAILED DESCRIPTION OF THE INVENTION

Given the generally poor long term prognoses for ovarian cancer there is an urgent need for new and particularly efficacious agents that target heretofore unapproved pathways for treatment. There are currently no officially indicated antiestrogens or selective estrogen receptor degraders (SERDs) FDA approved for the treatment of ovarian cancer making the need for a relatively non-toxic, well tolerated drug effective through the estrogen receptor a particularly exciting and important breakthrough. RAD1901 was found to inhibit tumor growth and/or drive tumor regression in breast cancer xenograft models, regardless of ESR1 mutation status and prior endocrine therapy. In the examples provided herein, RAD1901 was shown to be effective to inhibit tumor growth and unexpectedly drive tumor regression in certain PDX models (mice bearing a patient derived ovarian cancer xenograft) at a daily oral dosage of 30 mg/kg or 60 mg/kg.

As used herein, RAD1901 has the following structure,

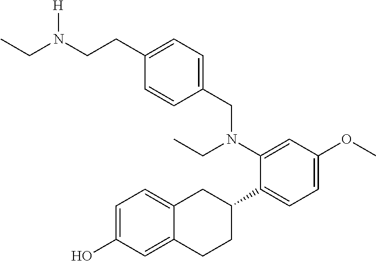 [SMILES: CCNCCc1ccc(CN(CC)c2cc(OC)ccc2[C@@H]2CCc3cc(O)ccc3C2)cc1]

including salts, isomers, solvates (e.g., hydrate), and prodrugs thereof.

In certain embodiments, methods are provided for treating ovarian cancer or tumor in a subject in need thereof comprising administering to the subject a therapeutically effective amount of RAD1901 as disclosed herein (e.g., or a salt or isomer or solvate (e.g., hydrate) or prodrug thereof).

In certain embodiments, methods are provided for inhibiting growth or producing regression of ovarian cancer or tumor in a subject in need thereof by administering to the subject a therapeutically effective amount of RAD1901 as disclosed herein (e.g., or a salt or isomer or solvate (e.g., hydrate) or prodrug thereof).

In certain embodiments of the methods disclosed herein, the ovarian cancer or tumor is ERα positive.

In certain embodiments of the methods disclosed herein, the salt of RAD1901 is RAD1901 dihydrochloride having the following structure:

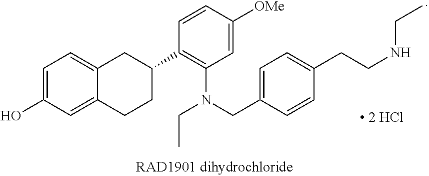 [SMILES: CCNCCc1ccc(CN(CC)c2cc(OC)ccc2[C@@H]2CCc3cc(O)ccc3C2)cc1 ; "."]

“Treating” of an ovarian cancer or tumor as used herein refers to achievement of one or more therapeutic benchmarks, including, without limitation, slowing or halting of tumor growth, driving tumor regression, reduction or cessation of one or more symptoms, reducing chances of having ovarian cancer or tumor, reducing the possibility or prolonging the time for a re-occurrence of ovarian cancer or tumor, etc.

“Inhibiting growth” of an ovarian cancer or tumor as used herein refers to slowing the rate of tumor growth, or halting tumor growth entirely.

“Tumor regression” or “regression” of an ovarian cancer or tumor as used herein refers to reducing the maximum size of the ovarian cancer or tumor.

In certain embodiments, administration of RAD1901 as disclosed herein (e.g., or a salt or isomer or solvate (e.g., hydrate) or prodrug thereof) to a subject having an ovarian cancer or tumor may result in a decrease in the ovarian cancer or tumor size versus baseline (i.e., size prior to initiation of treatment), or even eradication or partial eradication of the ovarian cancer or tumor. Accordingly, certain embodiments of the methods disclosed herein result in reducing the ovarian cancer burden or tumor size of one or more tumors versus pre-treatment baseline.

“Tumor” as used herein is malignant tumor, and is used interchangeably with “cancer.”

Tumor growth inhibition or regression may be localized to a single tumor or to a set of tumors within a specific tissue or organ, or may be systemic (i.e., affecting tumors in all tissues or organs).

As RAD1901 is known to preferentially bind ERα versus estrogen receptor beta (ERβ), unless specified otherwise, estrogen receptor, estrogen receptor alpha, ER-α, ERα, ER, wild-type ER-α, and ESR1 are used interchangeably herein. “Estrogen receptor alpha” or “ERα” is encoded by the gene ESR1. An ovarian cancer or tumor that is “positive for estrogen receptor alpha,” “ERα-positive,” “ER+,” or “ERα+” as used herein refers to an ovarian cancer or tumor in which one or more cells express at least one isoform of ERα. In certain embodiments, these cells overexpress ERα.

In certain embodiments of the methods disclosed herein, the ovarian cancer or tumor is resistant to a drug selected from the group consisting of anti-estrogens (e.g., tamoxifen or fulvestrant), aromatase inhibitors (e.g., aromasin), and combinations thereof.

In general, the methods disclosed herein are applicable to the treatment of an ovarian-originating tumor whether that tumor is located in the ovary or outside of the ovary (including elsewhere in the body) or both. For example, in some embodiments the primary ovarian cancer or tumor of the subject has already been surgically removed (e.g., by tumor excision or organ excision) and the subject is treated for 1) remaining metastatic ovarian cancer or tumor outside of one or more of her ovaries; and/or 2) reducing the possibility or prolonging the time for a re-occurrence. In some embodiments, the subject treated still has the primary ovarian cancer or tumor in one or more ovaries when the therapy is initiated.

In certain embodiments of the methods disclosed herein, RAD1901 is administered prophylactically to women deemed to be at high risk for ovarian cancer or tumor through one or more markers, characteristics, history or features that risk is ascertained. In such instances, the subject is medically determined to be at an elevated risk for ovarian cancer or tumor and RAD1901 is given to reduce the chances of the woman getting ovarian cancer or tumor.

In certain embodiments of the methods disclosed herein, the ovarian cancer or tumor being targeted is an epithelial ovarian cancer or tumor. In certain embodiments of the methods disclosed herein, the ovarian cancer or tumor is an epithelial ovarian cancer or tumor with a high grade serous, low grade serous, serous, endometrioid, mucinous, clear cell, primary peritoneal, brenner tumours, borderline tumours, or of undifferentiated type. In some embodiments the ovarian cancer to be treated is a stromal cancer or tumor. In some embodiments the ovarian cancer to be treated is an ovarian sarcoma.

In certain embodiments of the methods provided herein, the methods further comprise a step of determining whether a patient has an ovarian cancer or tumor expressing ERα prior to administering RAD1901 or a salt or solvate thereof, for example the bis-hydrochloride salt (2HCls per molecule of RAD1901). In certain embodiments the tumor to be treated expresses ER in >90%, >80%, >70%, >60%, >50%, >40%, >30%, >20%, >10%, >5%, or >1% of the cells in the ovarian cancer or tumor.

A therapeutically effective amount of RAD1901 for use in the methods disclosed herein is an amount that, when administered over a particular time interval, results in achievement of one or more therapeutic benchmarks (e.g., slowing or halting of tumor growth, driving tumor regression, cessation or reduction of one or more symptoms, reducing chances of having ovarian cancer or tumor, reducing the possibility or prolonging the time for a re-occurrence of ovarian cancer or tumor, etc.).

Examples of therapeutically effective amounts of RAD1901 for use in the methods disclosed herein include, without limitation, about 150 mg to about 2,000 mg, about 200 mg, about 400 mg, about 500 mg, or about 1,500 mg. More particularly the RAD1901 may be administered in a daily dosage amount of about 100 mg to about 1,000 mg, about 200 mg, about 400 mg, about 500 mg, about 600 mg, about 800 mg, or about 1,000 mg. In certain embodiments of the methods disclosed herein, the RAD1901 administered is a bis-hydrochloride of RAD1901 (2 HCls per molecule of RAD1901).

RAD1901 disclosed herein (and/or salts or isomers or solvates or prodrugs thereof) for use in the presently disclosed methods can be formulated into unit dosage forms, which are physically discrete units suitable as unitary dosage for subjects undergoing treatment. Each unit dosage contains a therapeutically effective amount of RAD1901, optionally in association with a suitable pharmaceutical carrier. The unit dosage form can be for a single daily dose or one for multiple daily doses (e.g., about 1 to 4 or more times q.d.). When multiple daily doses are used, the unit dosage form can be the same or different for each dose. In certain embodiments, RAD1901 disclosed herein may be formulated for controlled release.

RAD1901 disclosed herein (and/or salts or isomers or solvates or prodrugs thereof) for use in the presently disclosed methods can be formulated into a pharmaceutical composition as any the active ingredient. The pharmaceutical composition of RAD1901 may further comprises a physiologically acceptable carrier (also referred to as a pharmaceutically acceptable carrier or solution or diluent). Such pharmaceutical compositions are prepared in accordance with acceptable pharmaceutical procedures such as described in Remington's Pharmaceutical Sciences, 17th edition, ed. Alfonso R. Gennaro, Mack Publishing Company, Eaton, Pa. (1985), which is incorporated herein by reference.

The term “pharmaceutically acceptable carrier” refers to a carrier that does not cause an allergic reaction or other untoward effect in subjects to whom it is administered and are compatible with the other ingredients in the formulation. Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the therapeutic agent.

RAD1901 in a free base form can be converted into a salt by conventional methods. The term “salt” used herein is not limited as long as the salt is formed with RAD1901 in a free base and is pharmacologically acceptable; preferred examples of salts include a hydrohalide salt (for instance, hydrochloride, hydrobromide, hydroiodide and the like), an inorganic acid salt (for instance, sulfate, nitrate, perchlorate, phosphate, carbonate, bicarbonate and the like), an organic carboxylate salt (for instance, acetate salt, maleate salt, tartrate salt, fumarate salt, citrate salt and the like), an organic sulfonate salt (for instance, methanesulfonate salt, ethanesulfonate salt, benzenesulfonate salt, toluenesulfonate salt, camphorsulfonate salt and the like), an amino acid salt (for instance, aspartate salt, glutamate salt and the like), a quaternary ammonium salt, an alkaline metal salt (for instance, sodium salt, potassium salt and the like), an alkaline earth metal salt (magnesium salt, calcium salt and the like) and the like. In addition, hydrochloride salt, sulfate salt, methanesulfonate salt, acetate salt and the like are preferred “pharmacologically acceptable salt” of RAD1901 for the methods disclosed herein.

Isomers of RAD1901 disclosed herein (e.g., geometric isomers, optical isomers, rotamers, tautomers, and the like) can be purified using general separation means, including for example recrystallization, optical resolution such as diastereomeric salt method, enzyme fractionation method, various chromatographies (for instance, thin layer chromatography, column chromatography, glass chromatography and the like) into a single isomer. The term “a single isomer” herein includes not only an isomer having a purity of 100%, but also an isomer containing an isomer other than the target, which exists even through the conventional purification operation. A crystal polymorph sometimes exists for RAD1901 or a salt thereof, and all crystal polymorphs thereof are included in the present invention. The crystal polymorph of RAD1901 may be a single type of crystal polymorph or a mixture of multiple crystal polymorphs of RAD1901.

In certain embodiments, RAD1901 is in a prodrug form which will be converted (e.g., via oxidation or hydrolysis) to an active form of RAD1901.

In certain embodiments, the methods provided herein further comprise gene profiling the subject, wherein the gene to be profiled is one or more genes selected from the group consisting of ABL1, AKT1, AKT2, ALK, APC, AR, ARID1A, ASXL1, ATM, AURKA, BAP, BAP1, BCL2L11, BCR, BRAF, BRCA1, BRCA2, CCND1, CCND2, CCND3, CCNE1, CDH1, CDK4, CDK6, CDK8, CDKN1A, CDKN1B, CDKN2A, CDKN2B, CEBPA, CTNNB1, DDR2, DNMT3A, E2F3, EGFR, EML4, EPHB2, ERBB2, ERBB3, ESR1, EWSR1, FBXW7, FGF4, FGFR1, FGFR2, FGFR3, FLT3, FRS2, HIF1A, HRAS, IDH1, IDH2, IGF1R, JAK2, KDM6A, KDR, KIF5B, KIT, KRAS, LRP1B, MAP2K1, MAP2K4, MCL1, MDM2, MDM4, MET, MGMT, MLL, MPL, MSH6, MTOR, MYC, NF1, NF2, NKX2-1, NOTCH1, NPM, NRAS, PDGFRA, PIK3CA, P1K3R1, PML, PTEN, PTPRD, RARA, RB1, RET, RICTOR, ROS1, RPTOR, RUNX1, SMAD4, SMARCA4, SOX2, STK11, TET2, TP53, TSC1, TSC2, and VHL.

In certain embodiments, the methods provided herein further comprise adjusting the dosage of RAD1901 disclosed herein (and/or salt (e.g., HCl salt) or isomer or solvate (e.g., hydrate) or prodrugs thereof) comprising:
(1) administering a first dosage of RAD1901 disclosed herein (and/or salt (e.g., HCl salt) or isomer or solvate (e.g., hydrate) or prodrugs thereof) (e.g., about 350 to about 500 mg/day) for 3, 4, 5, 6, or 7 days;
(2) detecting estradiol-ER binding activity, for example using FES-PET imaging as disclosed herein; wherein:
   (i) if the ER binding activity is not detectable or is below a predetermined threshold level, continuing to administer the first dosage (i.e., maintain the dosage level); or
   (ii) if the ER binding activity is detectable or is above a predetermined threshold level, administering a second dosage that is greater than the first dosage (e.g., the first dosage plus about 50 to about 200 mg) for 3, 4, 5, 6, or 7 days, then proceeding to step (3);
(3) detecting estradiol-ER binding activity, for example using FES-PET imaging as disclosed herein; wherein
   (i) if the ER binding activity is not detectable or is below a predetermined threshold level, continuing to administer the second dosage (i.e., maintain the dosage level); or
   (ii) if the ER binding activity is detectable or is above a predetermined threshold level, administering a third dosage that is greater than the second dosage (e.g., the second dosage plus about 50 to about 200 mg) for 3, 4, 5, 6, or 7 days, then proceeding to step (4);
(4) repeating the steps above through a fourth dosage, fifth dosage, etc., until no ER binding activity is detected.

Certain embodiments of the methods disclosed herein further include the use of PET imaging to detect and/or dose ER sensitive or ER resistant cancers.

The term “and/or” as used herein includes both the “and” case and the “or” case.

Certain embodiments of the methods disclosed herein further comprises administering to the subject a therapeutically effective amount of one or more second therapeutic agents selected from the group consisting of CDK4 and/or CDK6 inhibitor(s) (e.g., ribociclib, abemaciclib and palbociclib), taxane (e.g., paclitaxel, albumin bound paclitaxel (e.g., abraxane), docetaxel, cabazitaxel), altretamine, capecitabine, etoposide, gemcitabine, ifosfamide, irinotecan, doxorubicin, liposomal doxorubicin, mephalen, pemetrexed, topotecan, vinorelbine, and PI3K inhibitors (e.g., idelalisib, perifosine, buparlisib, duvelisib, alpelisib, TGR1202, copanlisib, px-866, dactolisib, RP6530, SF1126, INK1117, pictilisib, XL147, XL765, palomid 529, ZSTK474, PWT33597, CUDC 907, ME401, IPI549, IC87114, TG100-115, CAL263, RP6503, PI103, GNE477, AEZS136). When more than one therapeutic agents are administered, the multiple therapeutic agents may be administered at the same time or substantially at the same time as one or more combined or separate pharmaceutical composition. Alternatively, the more than one therapeutic agents may be administered at different time by one controlled release pharmaceutical composition or by separate pharmaceutical composition.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Materials and Methods

RAD1901 used in the examples below was (6R)-6-(2-(N-(4-(2-(ethylamino)ethyl)benzyl)-N-ethylamino)-4-methoxyphenyl)-5,6,7,8-tetrahydronaphthalen-2-ol dihydrochloride, manufactured by IRIX Pharmaceuticals, Inc. (Florence, S.C.). RAD1901 was stored as a dry powder, formulated for use as a homogenous suspension in 0.5% (w/v) methylcellulose in deionized water, and for animal models was administered p.o.

### In Vivo Xenograft PDX Models

All mice were housed in pathogen-free housing in individually ventilated cages with sterilized and dust-free bedding cobs, access to sterilized food and water ad libitum, under a light dark cycle (12-14 hour circadian cycle of artificial light) and controlled room temperature and humidity.

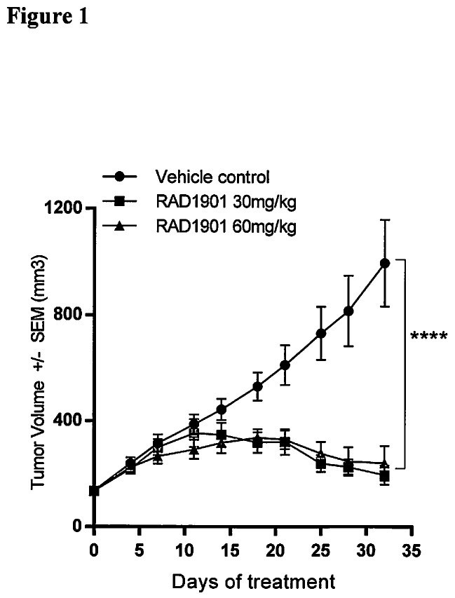

The PDX shown in FIG. 1 was the OV-10-0050 ovarian cancer tumor, derived from an ovarian adenocarcinoma from a 48 year woman. The tumor was grade 3 and positive for cytokeratin indicating an ovarian epithelial type. The tumor also stained positive for ER and an ER target gene (progesterone receptor). The OV-10-0050 PDX model was established from viable human tumor tissue that had been serially passaged in animals (female Balb/c nude mice) a limited number of times to maintain tumor heterogeneity. When tumors reached the appropriate Tumor Volume Initiation (TVI) range (150-250 mm³), animals were randomized into treatment and control groups and dosing initiated (Day 0, 8-10 subjects in each group); animals in all studies followed individually throughout each experiment. Initial dosing began Day 0; animals in all groups were dosed by weight (0.01 mL per gram; 10 ml/kg). Each group was treated with vehicle (control, p.o./q.d. to the endpoint), or RAD1901 (30 or 60 mg/kg of the subject, p.o./q.d. to the endpoint) as specified from day 0. Animals in RAD1901 60 mg/kg group were taken done on Day 32 due to weakness and heavy body weight loss. The treatment period of the remained animals (vehicle and RAD1901 30 mg/kg groups) lasted up to 42 days.

### Tumor Measurements

Tumor sizes were measured twice per week in two dimensions using a caliper, and the volume was expressed in mm³ using the formula: V=0.5 a×b² where a and b were the long and short diameters of the tumor, respectively. The tumor size was then used for calculations of tumor growth inhibition (TGI) value.

TGI was calculated for each group using the formula: TGI (%)=[1−(Ti−T0)/(Vi−V0)]×100; Ti is the average tumor volume of a treatment group on a given day, T0 is the average tumor volume of the treatment group on the day of grouping, Vi is the average tumor volume of the vehicle control group on a given day with Ti, and V0 is the average tumor volume of the vehicle group on the day of grouping.

### Statistical Analysis

Summary statistics, including mean and the standard error of the mean (SEM), are provided for the tumor volume of each group at each time point.

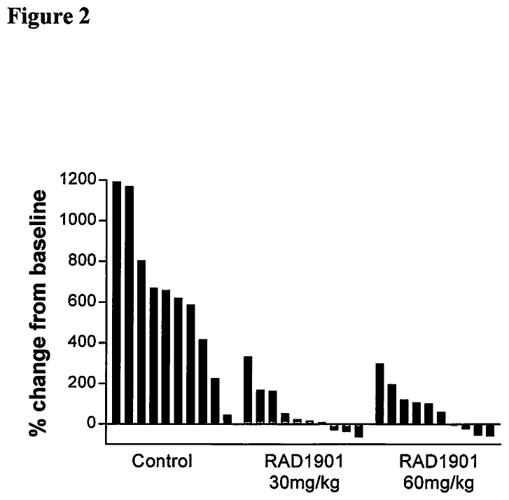

A one-way ANOVA was performed to compare tumor volume among RAD1901-treted groups compared to the vehicle-treated group on day 32 of treatment. All data were analyzed using Graphpad Prism. p<0.05 was considered to be statistically significant. As can be seen in FIG. 1, RAD1901 was efficacious (resulted in about 80% tumor growth inhibition, p-value>0.0001) and had approximately equal activity at both dose groups. Tumor sizes of individual animals on Day 32 show that RAD1901 resulted in tumor regressions in some animals treated in both 30 mg/kg and 60 mg/kg groups (FIG. 2).

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

1. A method of inhibiting tumor growth or producing tumor regression in a subject having ovarian cancer or tumor comprising administering to said subject a therapeutically effective amount of RAD1901 having the structure: 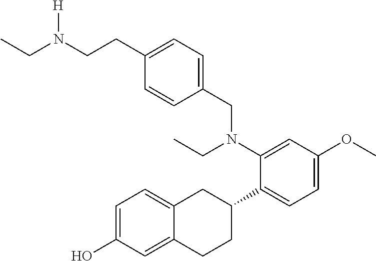 [SMILES: CCNCCc1ccc(CN(CC)c2cc(OC)ccc2[C@@H]2CCc3cc(O)ccc3C2)cc1]
or a salt or solvate thereof.

2. The method of claim 2, wherein said ovarian cancer or tumor expresses ERα.

3. The method of claim 1, wherein said ovarian cancer or tumor comprises an epithelial ovarian cancer.

4. The method of claim 1, wherein said ovarian cancer or tumor comprises a stromal ovarian cancer.

5. The method of claim 1, wherein said ovarian cancer or tumor comprises an ovarian sarcoma cancer.

6. The method of claim 1, wherein the cancer or tumor is a metastatic cancer.

7. The method of claim 1, wherein RAD1901 is administered at a daily oral dose of between 100 and 1,000 mg.

8. The method of claim 7, where the daily dose is 400 mg.

9. The method of claim 1, wherein the therapeutically effective amount is 150 mg to 2,000 mg.

10. The method of claim 9, wherein the therapeutically effective amount is 200 mg, 400 mg, or 500 mg.

11. The method of claim 1, wherein said administering further comprises the administration of a taxane.

12. The method of claim 1, wherein the ovarian cancer or tumor is resistant to a drug selected from the group consisting of anti-estrogens, aromatase inhibitors, and combinations thereof.

13. The method of claim 12, wherein the anti-estrogen is tamoxifen or fulvestrant.

14. The method of claim 12, wherein the aromatase inhibitors is aromasin.

15. The method of claim 1, further comprising administering to the subject a therapeutically effective amount of one or more second therapeutic agents selected from the group consisting of CDK4 and/or CDK6 inhibitors.

16. The method of claim 15, wherein the CDK4 and/or CDK6 inhibitors are selected from the group consisting of ribociclib, abemaciclib and palbociclib.

17. The method of claim 1, wherein one or more ovarian cancer or tumor of the subject has been surgically removed before the initiation of the administration of RAD1901.

18. The method of claim 1, wherein the subject has an elevated risk of having ovarian cancer or tumor, and the administration of RAD1901 reduces the chances of the subject getting the ovarian cancer or tumor.
